(19) United States
(12) Patent Application Publication
(10) Pub. No.: US 2007/0089759 A1
(43) Pub. Date: Apr. 26, 2007

(54) Hair treatment method and apparatus to minimize contact of toxic compositions with scalp and to facilitate post-treatment chemical removal

(76) Inventor: James S. Winterstein, Phoenix, AZ (US)

Correspondence Address: Tod R. Nissle, Esq.;TOD R. NISSLE, P.C., P.O. Box 55630, Phoenix, AZ, 85078, US

(21) Appl. No.: 10/782,333

(22) Filed: Feb. 19, 2004

(51) Int. Cl. A61K 8/18; A45D 2/00

(52) U.S. Cl. 132/208; 132/222

(57) ABSTRACT

An improved hair treatment method and apparatus minimizes the contact of toxic compositions with the scalp and facilitates the post-treatment removal of such compositions. The method utilizes a thin elastic strip of material that is provided with a fastening system that functions to maintain hair treatment chemicals and other chemicals away from the scalp and that functions to remove excess quantities of such chemicals from a customer's hair at the conclusion of a hair treatment.

This invention pertains to hair treatment.

More particularly, the invention pertains to a method and apparatus for coloring or otherwise treating hair on an individual's head while minimizing the risk that toxic chemicals will contact the skin on the head.

A variety of apparatus is known for coloring, perming, or otherwise treating the hair on an individual's head. One common type of apparatus consists of strips of metal foil or of a polymer material. In use, one of the strips is positioned with one end of the strip adjacent an individual's head. Selected strands of hair are laid on top of the strip. A liquid hair treatment chemical composition is applied to the hair and the strip is folded over the stransds of hair to allow the composition to act on the strands. The liquid composition can have a water-like viscosity, but often is more viscous and is like a thick syrup. After a predetermined period of time has passed, the strip is unfolded and is removed from the hair. A first end of the strip includes a VELCRO™ strip for attaching the strip adjacent the head of the individual and for attaching the other end of the strip to the first end when the strip is folded. See, for example, International Patent Application Number PCT/GB02/03256.

The conventional chemical application strips discussed above are widely used, but have several disadvantages. First, the strips that include a VELCRO fastener that is attached adjacent on individual's head tend to bleed or wick chemical to a location adjacent and in contact with the skin on the individual's head. Since the chemicals used in treating hair are often toxic, such “wicking” is particularly undesirable. Second, when a VELCRO fastener is pulled free from the head, the fastener ordinarily moves downwardly and creates an opening between the fastener and the head that further promotes contact of chemical with skin on the head. Third, when the strip is removed, the strip leaves a significant amount of chemical on the hair strands that were treated with the chemical, necessitating further clean up procedure to remove the chemical from the hair.

Accordingly, it would be highly desirable to provide an improved hair treatment method and apparatus that would minimize the quantity of hair treament chemical that contact the scalp and would minimize the quantity of residual chemical remaining on the individual's hair when treatment with the chemical is complete.

Therefore, it is a principal object of the invention to provide an improved hair treatment method and apparatus.

A further object of the invention is to provide an improved hair coloing treatment method and apparatus that reduces the risk that toxic hair treatment chemicals will contact the scalp of an individual.

Another object of the invention is to provide an improved hair coloring treatment method and apparatus that facilitates the removal of excess treatment chemicals from an individual's hair at the conclusion of the treatment.

These and other, further and more specific objects and advantages of the invention will be apparent to those skilled in the art from the following detailed description thereof, taken in conjunction with the drawings, in which:

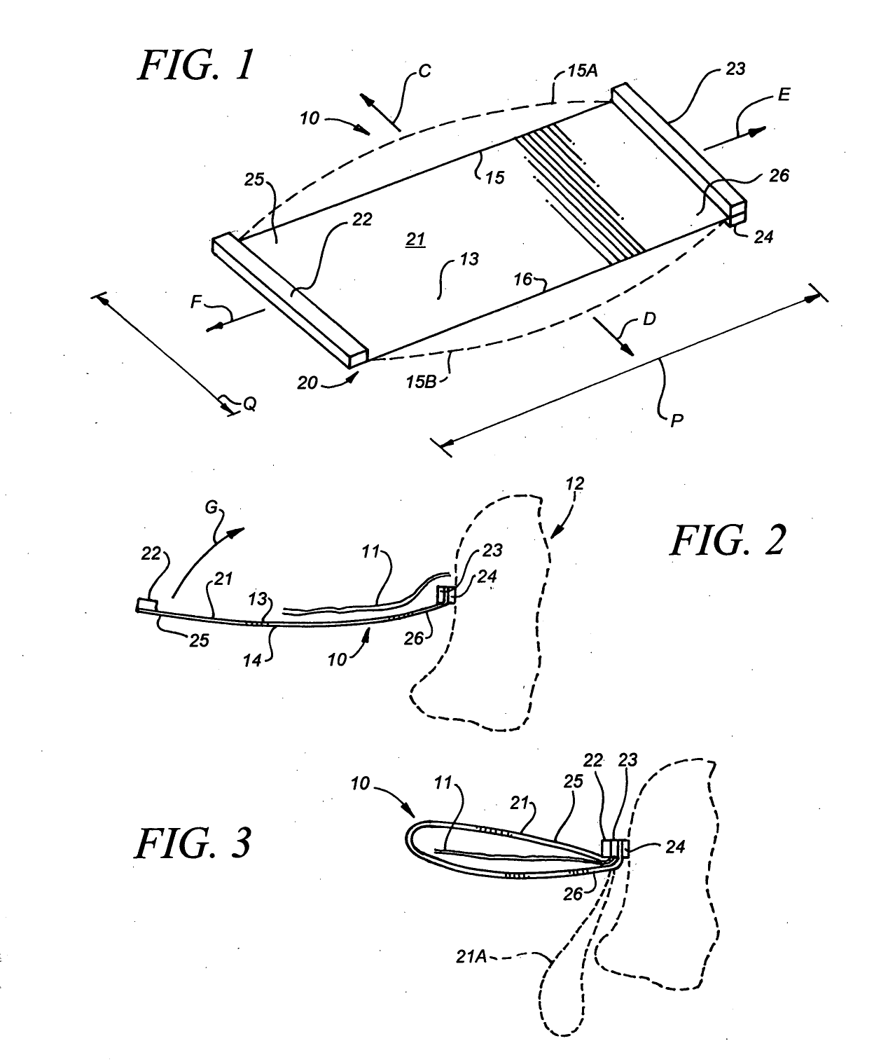

FIG. 1 is a perspective view illustrating hair treatment apparatus constructed in accordance with the principles of the invention;

FIG. 2 is a side view illustrating the mode of operation of the hair treatment apparatus of FIG. 1;

FIG. 3 is a side view further illustrating the mode of operation of the hair treatment apparatus of FIG. 1;

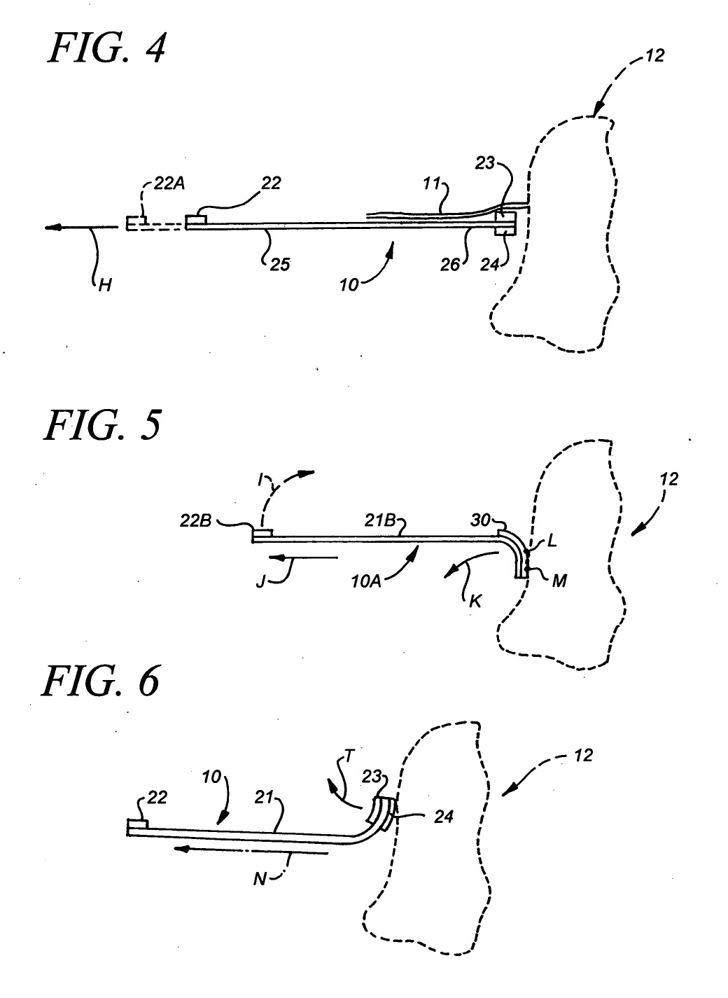

FIG. 4 is a side view still further illustrating the mode of operation of the hair treatment apparatus of FIG. 1;

FIG. 5 is a side view illustrating the mode of operation of prior art hair treatment apparatus;

FIG. 6 is a side view illustrating the mode of operation of the hair treatment apparatus of FIG. 1 during removal of the apparatus from the head of an individual; and,

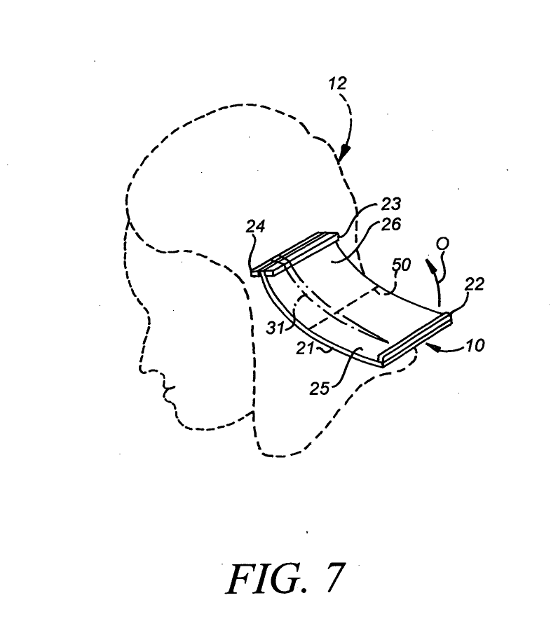

FIG. 7 is a perspective view further illustrating the use of the hair treatment apparatus of FIG. 1.

Briefly, in accordance with the invention, I provide an article of manufacture comprising a hair treatment apparatus. The hair treatment apparatus comprises a thin strip of liquid impermeable polymeric material. The strip has a first side and a second side which have first and second surfaces, respectively; a first end and a second end; a length dimension and a width dimension; a first fastening member on the first side at the first end; a second fastening member on the first side at the second end, the first and second fastening members being detachably attachable to one another, the second member being shaped and dimensioned to remove chemical composition from hair strands drawn over the second fastening member; and, a third fastening member on the second side at the second end to secure the second end to a selected location on the head such that when the strip is pulled in a direction away from and generally normal to the head, the third fastening member is pulled upwardly away from the head.

In a further embodiment of the invention, I provide an improved method for treating selected strands of hair on a head with a liquid chemical composition. The improved method includes the step of providing a thin elastic pliable strip of liquid impermeable polymeric material. The strip includes a first side and a second side which have first and second surfaces, respectively; a first end and a second end; a length dimension and a width dimension; a cleaning member on the first side at the second end, the cleaning member shaped and dimensioned to remove chemical composition from hair strands drawn over the cleaning member; and, securing the second end at a selected location on the head; selecting a plurality of hair strands on the head and placing the hair strands in an orientation extending over and contacting a portion of the cleaning member and the first side; applying the liquid chemical composition to the plurality of hair strands; and, displacing the strip in a direction away from the head such that the cleaning member is pulled along the hair strands and removes at least some of the liquid chemical composition from the hair strands.

In another embodiment of the invention, I provide an improved method for treating selected strands of hair on a head with a liquid chemical composition. The improved method includes the steps of providing a thin elastic pliable strip of liquid impermeable polymeric material. The strip includes a first side and a second side which have first and second surfaces, respectively; a first end and a second end; a length dimension and a width dimension; a first fastening member on the first side at the first end; a second fastening member on the first side at the second end, the first and second fastening members being detachably attachable to one another, the second member shaped and dimensioned to remove chemical composition from hair strands drawn over the second fastening member; a third fastening member on the second side at the second end to secure the second end to a selected location on the head such that when the strip is pulled in a direction away from and generally normal to the head, the third fastening member is pulled upwardly away from the head. The improved method also includes the steps of securing the third fastening member to the selected location on the head such that the second fastening member is above the third fastening member; selecting a plurality of hair strands on the head and placing the hair strands in an orientation extending over and contacting a portion of the second fastening member and the first side; applying the liquid chemical composition to the plurality of hair strands; folding the pliable strip and securing together the first and second fastening members; detaching the first fastening member from the second fastening member; and, pulling the first end in a direction away from the head such that the third fastening member is pulled upwardly away from the head, and the second fastening member is pulled along the hair strands and removes at least some of the liquid composition from the hair strands.

Turning now to the drawings, which depict the presently preferred embodiments of the invention for the purpose of illustrating the practice thereof and not by way of limitation of the scope of the invention and in which like reference characters refer to corresponding elements throughout the several views, FIG. 1 illustrates hair treatment apparatus constructed in accordance with the principles of the invention and generally indicated by reference character 10. Apparatus 10 includes a thin elastic pliable strip 21 of liquid impermeable polymeric material having a first top side and a second bottom side. The top side includes surface 13. The bottom side includes surface 14. Strip 21 includes a first end 25 and a second end 26; a length P, and, a width Q that normally, but not necessarily, is less than the length P. Strip 21 also includes a first fastening member 22 on the first side 13 at end 25; a second fastening member 23 on the first side 13 at end 26; and a third fastening member 24 on side 14 at end 26. Fastening members 22 and 23 are constructed to be detachably secured to one another. Any construction of members 22 and 23 that achieves this function can be utilized. For example, member 22 can be constructed of VELCRO hook material and member 23 can be constructed of VELCRO loop material. Or, member 22 can be coated with a releasable adhesive than can be detachably secured to member 23.

Member 24 is constructed to be detachably secured to or adjacent the head of an individual. Any construction of member 24 that achieves this function can be utilized. For example, member 24 can be constructed of VELCRO hook material to engage hair at a location adjacent an individual's scalp. Or, member 24 can include an adhesive that releasably adheres to the scalp or to hair adjacent the scalp.

Apparatus 10 is preferably, but not necessarily, elastic such that it can be stretched along its length in the directions indicated by arrows E and F, and, such that it can be stretched laterally in the directions indicated by arrows C and D to expand, for example, edge 15 to the position indicated by dashed line 15A and edge 16 to the position indicated by dashed line 15B. Strip 21 is presently preferably comprised of latex sheeting, but any other desired material can be utilized. The latex sheeting is preferably silky smooth on both sides, is durable and tear-resistant; is chemical resistant, and retains its structural integrity under high-pressure. The thickness of the latex sheeting can vary as desired, but is preferably from 0.004 inch to 008 inch.

For sake of the following description, member 23 is comprised of loop VELCRO material, and members 22 and 24 are comprised of hook VELCRO material. The shape and dimension of each member 22 to 24 can vary as desired, and each member 22 to 24 can, if desired, be comprised of two or more separate or adjacent pieces of member.

In use, member 24 is pressed against the head 12 of an individual. The VELCRO hook material engages hair adjacent the scalp. When member 24 is pressed against the head 12, member 23 presently preferably is above member 24 and is spaced completely or partially away from scalp. When member 23 is in the position shown in FIGS. 2, 3, 7, it tends to function as a dam or impediment to fluid hair coloring chemicals or other hair treatment chemicals that are on surface 13, and, therefore tends to prevent such chemicals from reaching and contacting the scalp, in part because member 23 is spaced away from the scalp by member 24. This dam function is an important function of member 23.

After apparatus 10 is positioned in the manner shown in FIG. 2 and strands of hair are positioned on surface 13, a liquid chemical composition is applied to strands 11, apparatus 10 is folded to the position shown in FIG. 3, and member 22 is detachably secured to member 23. After a selected period of treatment time has passed, member 22 is detached from member 23 and member 22 and end 25 are grasped and pulled in a direction H that is generally normal to the head of individual 12. When apparatus 10 is so pulled, strip 21 stretches and exerts additional force on memebers 23 and 24 that functions to separate member 24 from its position adjacent head 12 and that tends to pull member 23 along strands 11. When member 23 moves along strands 11, it functions to absorb or remove residual chemical composition from strands 11.

As is illustrated in FIG. 6, when member 24 is pulled free from the head 12 of an individual by pulling strip 21 outwardly in the direction of arrow N, member 24 tends to peel upwardly and outwardly away from head 12 in the direction indicated by arrow T. This upward separation of member 24 functions, along with the spacing of member 23 away from the scalp, to minimize the quantity of hair treatment chemical that comes into contact with the scalp. In contrast, as shown in FIG. 5, when prior art apparatus 10A is pulled free from the head 12 of an individual, fastening member 30 peels off downwardly and outwardly in the direction of arrow K (so that point L separates from the head 12 before point M). Such downward peeling of member 30 tends to facilitate the wicking or bleeding of liquid chemical by member 30 into contact with the scalp.

FIG. 7 illustrates hair strands 31 positioned on strip 21 and extending over fold line 50 before apparatus 10 is folded upwardly in the direction of arrow) to attach member 22 to member 23.

Having set forth my invention in terms to enable those skilled in the art to understand and practice the invention and having set forth the presently preferred embodiments and uses thereof, I Claim:

1. An article of manufacture comprising a hair treatment apparatus comprising a thin strip of liquid impermeable polymeric material having
(a) a first side and a second side which have first and second surfaces, respectively;
(b) a first end and a second end;
(c) a length dimension and a width dimension;
(d) a first fastening member on said first side at said first end;
(e) a second fastening member on said first side at said second end said first and second fastening members being detachably attachable to one another, said second member shaped and dimensioned to remove chemical composition from hair strands drawn over said second fastening member; and,
a third fastening member on said second side at said second end to secure said second end to a selected location on the head such that when said strip is pulled in a direction away from and generally normal to the head, said third fastening member is pulled upwardly away from the head.

2. A method for treating selected strands of hair on a head with a liquid chemical composition, including
(a) providing a thin elastic pliable strip of liquid impermeable polymeric material having
(i) a first side and a second side which have first and second surfaces, respectively,
(ii) a first end and a second end,
(iii) a length dimension and a width dimension,
(iv) a cleaning member on said first side at said second end, said cleaning member shaped and dimensioned to remove chemical composition from hair strands drawn over said cleaning member,
(b) securing said second end at a selected location on the head;
(c) selecting a plurality of hair strands on the head and placing the hair strands in an orientation extending over and contacting a portion of said cleaning member and said first side;
(d) applying the liquid chemical composition to the plurality of hair strands;
(e) displacing said cleaning member in a direction away from the head such that said cleaning member is pulled along the hair strands and removes at least some of the liquid chemical composition from the hair strands.

3. A method for treating selected strands of hair on a head with a liquid chemical composition, including
(a) providing a thin elastic pliable strip of liquid impermeable polymeric material having
(i) a first side and a second side which have first and second surfaces, respectively,
(ii) a first end and a second end,
(iii) a length dimension and a width dimension,
(iv) a first fastening member on said first side at said first end,
(v) a second fastening member on said first side at said second end said first and second fastening members being detachably attachable to one another, said second member shaped and dimensioned to remove chemical composition from hair strands drawn over said second fastening member
(vi) a third fastening member on said second side at said second end to secure said second end to a selected location on the head such that when said strip is pulled in a direction away from and generally normal to the head, said third fastening member is pulled upwardly away from the head;
(b) securing said third fastening member to the selected location on the head such that said second fastening member is above said third fastening member;
(c) selecting a plurality of hair strands on the head and placing the hair strands in an orientation extending over and contacting a portion of said second fastening member and said first side;
(d) applying the liquid chemical composition to the plurality of hair strands;
(e) folding said pliable strip and securing together said first and second fastening members;
(f) detaching said first fastening member from said second fastening member;
(g) pulling said first end in a direction away from the head such that
(i) said third fastening member is pulled upwardly away from the head, and
(ii) said second fastening member is pulled along the hair strands and removes at least some of the liquid composition from the hair strands.
